(19) United States
(12) Patent Application Publication
(10) Pub. No.: US 2011/0027844 A1
(43) Pub. Date: Feb. 3, 2011

(54) METHOD FOR PRODUCING QUINONES

(75) Inventors: Masaaki Morikawa, Hokkaido (JP); Yasushi Oogo, Saitama (JP); Yoshio Suzuki, Tokyo (JP)

Correspondence Address: SUGHRUE MION, PLLC, 2100 PENNSYLVANIA AVENUE, N.W., SUITE 800, WASHINGTON, DC, 20037, US

(73) Assignee: NISSHIN PHARMA INC., Tokyo (JP)

(21) Appl. No.: 12/716,933

(22) Filed: Mar. 3, 2010

(30) Foreign Application Priority Data
Jul. 31, 2009 (JP) 2009-179141

(51) Int. Cl. C12P 7/26

(52) U.S. Cl. 435/148

(57) ABSTRACT

A method for conveniently and efficiently producing quinones, and particularly menaquinone, from a microorganism is provided. The present invention relates to a method for producing quinones, comprising culturing a microorganism that produces quinones in the presence of a porous carrier.

### BACKGROUND OF THE INVENTION

The present invention relates to a method for conveniently and efficiently producing large amounts of quinones, and preferably menaquinone.

### BACKGROUND ART

Various molecules exist in vivo with functions of maintaining the living body through interaction with each other or with the outer world. Among such molecules, many quinones, such as menaquinone, ubiquinone, plastoquinone and phylloquinone, are molecules involved in the energy production system of the electron transport system in vivo. Thus, these quinones are extremely important molecules. Quinones also function as coenzymes and have been revealed to exert antioxidative action and various pharmacological effects, and thus they are developed as pharmaceutical products or, further, as functional ingredients of foods. Therefore, Quinones constitute a compound group that has recently gained much attention.

These quinones are also contained in natural foods, but the amounts thereof are extremely low. Hence, it is difficult to gain the necessary and sufficient amounts thereof through daily food ingestion. Accordingly, quinones obtained by a chemical synthesis method or a fermentation method, or those extracted and purified from organisms and the like are condensed and then used, resulting in extremely high costs. Thus, most quinones are used as pharmaceutical products. Therefore, most of quinones cannot still be ingested daily in a convenient manner under the circumstances. Also, compounds having quinone skeletons are frequently fat-soluble and insoluble or hardly soluble in water, so that handling thereof for processing into foods and the like has been inconvenient.

Among the quinones, vitamins K can be further classified into vitamins K1 to K7 based on side chain differences. Among them, vitamins K1 and K2 exist in nature and vitamins K3 to K7 are obtained by artificial synthesis. Vitamin K1 (phylloquinone) is mainly contained in plants, and vitamin K2 (menaquinone) is mainly contained in animals and microorganisms. It has been known for a long time that vitamin K is involved in the blood coagulation system. However, only a trace amount of vitamin K is involved in the blood coagulation system, so that deficiency symptoms almost never occur. Hence, vitamin K has not attracted much attention. Recently, it has been revealed that vitamin K is important for the maintenance of bone mass. As the number of osteoporosis patients is increasing, interest in vitamin K is increasing. Large amounts of vitamin K are required for its involvement in the maintenance of bone mass, compared with the amounts required for involvement in the blood coagulation system. Prevention or treatment of osteoporosis requires several mg to several tens of mg of vitamin K per day.

The amount of quinones contained in an organism is low, and thus they cannot be easily extracted. Regarding menaquinone (vitamin K2), Bacillus subtilis var natto, a kind of a microorganism, Bacillus subtilis, is known to produce and secrete menaquinone outside the cells. Therefore, production of menaquinone using such property has been attempted. However, Bacillus subtilis var natto produces menaquinone in an amount as small as several mg/L at best. To supply the required menaquinone through direct ingestion of natto, as large an amount as a few hundred gram of menaquinone should be ingested, which is unrealistic. Hence, methods for addressing such problem have been proposed.

Patent document 1 discloses that Bacillus subtilis is cultured, vitamin K (menaquinone) produced within the cells is recovered before it is released extracellularly, and the prolonged effects of high concentrations thereof in plasma are exerted by ingesting the cells themselves. However, menaquinone is assumed to be present within the cells in this method, so that complicated procedures must be carried out, such as direct ingestion of the cells or recovery of menaquinone via disruption of the cells. Thus, the former procedure is unfavorable due to flavor issues, and the latter procedure is unfavorable due to cost. Also, Patent document 2 discloses a method for producing menaquinone, comprising preparing a medium for menaquinone-producing bacteria by culturing filamentous bacteria of Aspergillus with a medium comprising barley and/or a disrupted product of barley, glycosylating and then neutralizing the thus obtained culture solution, and culturing menaquinone-producing bacteria using the prepared solution. Patent document 3 discloses a method for producing menaquinone, comprising adding glycerin to a solution containing one or more types of soybean powder, defatted soybean powder and soymilk, and then culturing Bacillus subtilis var natto in said liquid medium with adjusted pH. However, these methods lead to high costs because of the use of special medium. Moreover, these methods are problematic in that large amounts of impurities remain in such medium, requiring great efforts for treatment or purification before and after culture.

[Patent document 1] JP Patent Publication (Kokai) No. 2001-175 A
[Patent document 2] JP Patent Publication (Kokai) No. 2001-333792 A
[Patent document 3] JP Patent Publication (Kokai) No. 2002-315594 A

### SUMMARY OF THE INVENTION

An object to be achieved by the present invention is to provide a method for conveniently and efficiently producing quinones, and particularly menaquinone, from a microorganism.

The present inventors have focused on the fact that Bacillus subtilis var natto producing menaquinone releases water soluble menaquinone, following which they conducted intensive studies thereon. Thus, they have discovered that menaquinone forms a complex with surfactin, becoming water soluble. As a result of focusing on such phenomenon and attempting culture under various conditions, they have discovered that when culture is carried out in the presence of a porous carrier, surprisingly, Bacillus subtilis var natto forms a biofilm on the porous carrier and produces and releases a large amount of menaquinone therewithin. Thus, they have completed the present invention.

The following inventions are encompassed herein.

(1) A method for producing quinones, comprising culturing a microorganism that produces quinones in the presence of a porous carrier.
(2) The method according to (1), wherein the microorganism is a microorganism forming a biofilm.
(3) The method according to (1) or (2), wherein the microorganism is a microorganism producing a biosurfactant.
(4) The method according to any one of (1) to (3), wherein the microorganism is a microorganism belonging to Bacillus.
(5) The method according to (4), wherein the microorganism is Bacillus subtilis.
(6) The method according to (5), wherein the microorganism is Bacillus subtilis var natto.
(7) The method according to any one of (1) to (6), further comprising adding a biosurfactant to a culture solution.
(8) The method according to any one of (1) to (7), wherein a quinone is menaquinone.
(9) The method according to any one of (1) to (8), wherein culture is carried out with aeration.

### EFFECT OF THE INVENTION

According to the present invention, quinones, and particularly menaquinone, from a microorganism can be conveniently produced in a highly efficient manner.

### BRIEF DESCRIPTION OF THE DRAWINGS

FIG. 1 is a graph showing differences in menaquinone yield resulting from the presence or the absence of a porous carrier.

FIG. 2 is a graph showing differences in menaquinone yield resulting from the presence or the absence of a biosurfactant.

FIG. 3 is a graph showing the relationship between the amount of a biofilm formed and menaquinone yield.

FIG. 4 is a graph showing differences in menaquinone yield resulting from aeration volumes.

FIG. 5 is a graph showing differences in menaquinone yield resulting from culture methods.

FIG. 6 is a graph showing that a culture solution can be condensed through aeration via a porous carrier.

### DETAILED DESCRIPTION OF THE PREFERRED EMBODIMENTS

Hereinafter, preferred embodiments of the present invention will be described specifically. The method of the present invention is characterized by culturing a microorganism producing quinones in the presence of a porous carrier, growing it so that it forms a larger amount of a biofilm, and thus causing the efficient production of a large amount of quinones.

In the present invention, the term “quinones” refers to whole compounds having quinone structures, and specifically, it preferably refers to such compounds having a certain activity to organisms. Examples of such quinones include ubiquinone, plastoquinone, menaquinone, phylloquinone, rhodoquinone and pyrroloquinoline quinone. Ubiquinone, phylloquinone and plastoquinone are present in the photosystem (electron transport system) in organisms. Menaquinone and phylloquinone are also known as vitamin K2 and vitamin K1, respectively. Regarding menaquinone, molecular species of menaquinone 1-14 are present based on differences in side chain structure. Menaquinone 7 (MK-7) is produced by Bacillus subtilis var natto.

Microorganisms to be used in the present invention are not particularly limited, as long as they have property of producing quinones. Examples of microorganisms having such property include microorganisms belonging to Flavobacterium, microorganisms belonging to Rhodopseudomonas, microorganisms belonging to Corynebacterium, microorganisms belonging to Brevibacterium, and microorganisms belonging to Bacillus. Bacillus subtilis is preferable among these microorganisms and particularly Bacillus subtilis var natto is most preferably used because the safety of the microorganisms and the culture products have been established. Regarding Bacillus subtilis, known strains, strains extracted from nature, mutants thereof, and the like can be used. For example, a B-1 wild strain isolated from Sagara oil field, Bacillus subtilis DB9011 strain, Bacillus subtilis MS-01 strain, Bacillus subtilis C-3102 strain, Bacillus subtilis BN strain, Bacillus subtilis NBRC3009 strain, Bacillus subtilis NBRC3025 strain, Bacillus subtilis NBRC3108 strain, Bacillus subtilis NBRC3336 strain, Bacillus subtilis 168 strain, Bacillus subtilis 3610 strain and Bacillus subtilis ATCC6633 strain can be used. Regarding Bacillus subtilis var natto, known strains, such as strains derived from commercially available “natto”, strains from commercially available Bacillus subtilis var natto such as Takahashi strain (Yuzo Takahashi Laboratory, Yamagata, Japan), Naruse strain (Kabushikigaisha Naruse Hakko Kagaku Kenkyusho (Naruse Fermentation Chemistry Laboratory), Tokyo, Japan), Miyagino strain (Yugengaisha Miyagino Natto Seizo-sho (Miyagino Natto Laboratory, Ltd.), Sendai, Japan), Asahi strain (Asahi Industries Co. Ltd., Tokyo, Japan), Nitto strain (Nitto Pharmaceutical Industries, Ltd., Kyoto, Japan) and Meguro strain (Meguro Institute Co., Ltd., Osaka, Japan) or strains deposited such as FERM BP-6713 strain and ATCC21332 strain, can be used herein.

Alternatively, even if microorganisms have no such property of producing quinones, microorganisms into which the property of producing quinones has been introduced by a known method, or microorganisms altered to be able to produce quinones, may also be used herein. Examples of the aforementioned method include a selection by crossing microorganisms, induction of mutation by radiation, chemical substances or the like, and gene transfer. For example, general Bacillus subtilis var natto that produces menaquinone 7 can be altered to produce menaquinone 4 by introducing a polyprenyl diphosphate dehydrogenase gene or the like thereinto.

Among the above microorganisms, preferable microorganisms have the property of increasing production of quinones by forming a biofilm on a porous carrier when cultured in the presence of the porous carrier, and further preferable microorganisms have the property of producing a biosurfactant.

The term “biofilm” refers to an aggregate of extracellular polysaccharides produced by microorganisms. It is known that within the biofilm microorganisms form a community, exhibiting properties that differ from those at the time of suspension culture or acquiring resistance to the external environment. The property of forming a biofilm may be conferred via introduction or alteration by a known method.

The term “biosurfactant” generally refers to various surfactant-like substances from microorganisms and known examples thereof include rhamnolipid, sophorolipid, surfactin and arthrofactin. These biosurfactants are advantageous in that they have no biological toxicity or environmental residual tendency as in the case of synthetic surfactants while retaining surface activity. When quinones are secreted extracellularly together with such biosurfactant, fat-soluble quinones acquire hydrophilicity. Such quinones are excellent in handleability and thus are preferred. The property of producing a biosurfactant may be conferred via introduction or alteration by a known method. Examples of such method include selection by crossing microorganisms, induction of mutation by radiation, chemical substances or the like, and gene transfer. A biosurfactant may be added from the outside into a culture environment. However, biosurfactants that are produced and secreted by microorganisms themselves are preferred since such biosurfactants can efficiently increase the production amounts of quinones. Also when a microorganism itself secretes a biosurfactant, quinones can be produced more efficiently through further addition of a biosurfactant to a culture solution. In such case, a biosurfactant is preferably added to a culture solution at a concentration ranging from 10 μM to 1000 μM and preferably ranging from 100 μM to 200 μM.

Examples of porous carriers to be used in the present invention include: inorganic compounds such as glass, quartz, ceramics, bentonite, zeolite, pumice, Al₂O₃, ZrO₂, TiO₂, MgO, SiO₂, CaCO₃ and zeolite; as well as organic compounds such as plastic (e.g. polystyrene, polypropylene, polyethylene, polyester, nylon, acryl, polycarbonate, polyurethane, fluorocarbon polymers such as polytetrafluoroethylene, methylpentene resin, phenol resin, melamine resin and epoxy resin), fibers made of polysaccharides or the like, and porous membranes such as nitrocellulose and nylon. Of these examples, porous carriers having properties appropriate for culture in the present invention, such as stability to acid or alkali, good air permeability and repellency are preferred.

The percentage of void of a porous carrier ranges approximately from 70% to 99%, preferably approximately from 75% to 97%, and more preferably approximately from 80% to 95%, for example. Furthermore, the pore size of a porous carrier ranges from approximately 2 μm to 2 mm and preferably approximately from 5 μm to 1 mm.

The microorganism of the present invention can efficiently produce quinones on a porous carrier having the above properties. Moreover, such porous carrier is materially stable, so that it never releases harmful chemical materials or the like into the culture solution. Furthermore, such porous carrier continues to keep the solid form, and is never soluble, for example. Hence, the porous carriers can be easily separated from the culture product containing quinones that are products by known procedures such as solid-liquid separation. Additionally, when a microorganism that forms a biofilm on a porous carrier is used, the biofilm also contains a large amount of menaquinone. Recovery of such menaquinone can be easily achieved by separating the porous carrier from the culture solution. Therefore, the production method of the present invention is an extremely advantageous method since the purification after culture in this production method can be easily carried out, compared with a method that involves dispersing, suspending, and dissolving special ingredients in a culture solution.

The term “culture product(s)” in this specification refers to a generic term for a culture solution or a biofilm containing quinones after quinones are produced by culture. Such culture products include, depending on the conditions such as environment for production, a culture solution alone, a biofilm alone or a mixture thereof, and these products subjected to steps such as purification and drying. Such culture products may also contain microorganisms.

A known method for culturing a microorganism may be appropriately selected and used depending on the assimilability of a microorganism to be used herein, environment for culture or production, production scale and the like. Natural or synthetic medium comprising liquid medium, solid medium, gelatin medium or the like can be used. Any medium may be used herein, as long as it contains a carbon source such as starch and glucose, a nitrogen source such as a meat extract and polypeptone, minerals such as sodium and potassium and the like. Synthetic liquid medium is preferably used because the thus generated quinones can be easily purified. The pH of medium may be appropriately determined in view of handleability and the like, as long as the conditions allow a microorganism of the present invention to be able to properly grow to produce quinones. In general, the pH ranges from 6.0 to 9.0 and preferably from 6.5 to 8.0.

Culture conditions can also be appropriately selected depending on types of microorganism to be used, microbial strains and medium composition. Such culture conditions are not particularly limited, as long as a microorganism to be used can grow to produce quinones under the conditions. In general, the culture temperature ranges from 20° C. to 45° C. and preferably from 30° C. to 42° C. The culture period from 6 hours to 14 days and preferably ranges from 20 hours to 4 days.

For efficient culture using a porous carrier in the present invention, static culture rather than shaking culture, is more preferably performed. The reason for this is unknown, but it may be associated with the fact that a microorganism forms a biofilm more efficiently on a porous carrier, quinones produced by the microorganism within the biofilm form complexes with biosurfactants, and then the complexes are accumulated at a high concentration. Examples of static culture include a method whereby static culture is carried out throughout the culture period from the initiation to the termination of culture and a method whereby first shaking culture is carried out, followed by a switch to static culture in the middle of the process. Both methods may be employed herein. In general, microorganisms are thought to consume energy for self-propagation during a logarithmic growth phase, following which they produce and secrete extracellularly various substances when they reach a stationary phase. Hence, shaking culture is performed until the initial stationary phase, and then a switch to static culture takes place to allow the microorganism to reach the stationary phase, so as to cause efficient production of quinones.

When culture, and particularly static culture, is carried out in the present invention, the culture solution is preferably aerated. At such time, further preferably a tubular porous carrier is used and aeration is performed via the porous carrier. By culturing while performing aeration via such porous carrier, the culture solution is adequately stirred, even in the case of static culture. Thus, usability of nutritional components is increased and the culture solution is condensed, so that a culture product containing quinones at a higher concentration can be obtained.

Furthermore, with the use of a porous carrier, culture is carried out continuously, and the culture product can be obtained efficiently. Specifically, the greater part of a biofilm adheres to a porous carrier and quinones are accumulated in the biofilm at a high concentration. After completion of culture, the porous carrier to which the biofilm has adhered is removed without discarding the culture solution, and then a new porous carrier is added to the culture solution. Through addition of new essential nutrients for culture, fresh culture can be initiated. Meanwhile, when the removed porous carrier is added to a new culture solution, another culture is also initiated, so that menaquinone will be produced in the culture solution. The use of such continuous culture method enhances production efficiency such that: 1) there is no need to discard the culture solution; 2) there is no need to newly add a microorganism; and 3) there is no need to perform a step of washing the culture vessels. Furthermore, such method is also an environmentally effective production method.

Recovery and purification of quinones from the culture product of the present invention can be appropriately carried out by known methods. In the present invention, quinones are recovered and purified from biofilms among culture products, so that quinones can be recovered at a higher concentration.

When quinones are purified from a culture solution, a liquid-liquid extraction method, an adsorption method, a distillation method, a chromatography method or the like can be employed. Furthermore, these methods may be combined. The liquid-liquid extraction method involves extracting and dispensing quinones with the use of an organic solvent such as hydrocarbon, alcohol, ether, ester or ketone. The adsorption method involves causing an adsorbent such as a carbon-based adsorbent to adsorb quinones, removing contaminants and then eluting quinones. The distillation method involves separation using different boiling points of quinones and contaminants. However, since quinones are denatured at high temperatures, a distillation method that can be implemented at low temperatures, such as a molecular distillation method, is preferable. The chromatography method involves separation based on interaction between quinones, and a solid-phase support and a mobile phase filling the column.

Since a biofilm adheres to a porous carrier, quinones in a biofilm are purified, for example, by removing a porous carrier from a culture layer and then separating the biofilm by a method that involves physically scraping off the biofilm from the carrier or that involves removing the biofilm in aqueous liquid by ultrasonication. When quinones are then purified, various methods for purifying quinones from the above culture solution can be applied to the thus separated biofilm.

Culture products obtained in the present invention may be directly added to pharmaceutical products, foods or feedstuff compositions, as long as they are produced using microorganisms whose safety has been particularly confirmed. The intake thereof can be appropriately determined in view of target effects, types of quinones contained and the contents thereof. For example, when a quinone is ubiquinone (coenzyme Q10) and it is ingested as a pharmaceutical composition, the daily intake for a patient with symptoms of congestive heart failure may be 30 mg. When the quinone is ingested by a healthy subject as a health food, the daily intake may be approximately 10 mg to 50 mg as an indication. Also, when a quinone is menaquinone (e.g., MK-4 or MK-7) and ingested as a pharmaceutical composition, the daily intake for a patient with hemorrhagic disorder may be 5 mg to 20 mg. When the quinone is ingested by a healthy subject as a health food for prevention of osteoporosis, daily intake may be approximately 30 mg to 100 mg as an indication.

When pharmaceutical compositions containing quinones or culture products obtained according to the present invention as active ingredients are prepared, they are generally prepared as preparations containing pharmaceutically acceptable carriers. Such pharmaceutical composition may be administered via oral, enteral or transanal route. Additives or other known functional ingredients may also be compounded.

Examples of formulations of pharmaceutical compositions include oral preparations such as tablets, capsules, granules, powders, syrups, dry syrups, solutions and suspensions, as well as enteral preparations such as inhalers and suppositories. Of these examples, oral preparations are preferable.

In the case of such formulation of a pharmaceutical composition, conventionally used additives such as an excipient, a disintegrator, a binder, a lubricant, a surfactant, alcohol, water, a water soluble polymer, a sweetener, a flavoring agent, an acidulant, or the like may be compounded in the above quinones or culture products depending on formulations. In addition, liquid preparations such as solutions and suspensions may be in forms so that they are dissolved or suspended in water or another appropriate medium immediately before dose. Alternatively, in the form of tablets and granules, the surfaces thereof may be coated by a known method.

In a pharmaceutical composition, the content of an active ingredient, a quinone or a culture product may differ depending on the formulations. The content may be generally within the range between 1% by mass and 99% by mass and preferably between 5% by mass and 80% by mass, based on dry mass. It is desired to be able to control daily dosage so that an adult can ingest the aforementioned daily intake.

When quinones or culture products obtained according to the present invention are added to food compositions, forms of the food compositions are not particularly limited. Examples of such food compositions include, in addition to health foods, functional foods, and specified health foods, all foods in which the above quinones or culture products can be compounded. Specifically, specified health foods may be formulated into various dosage forms, such as fluid diets (e.g., tube enteral nutrients), tablets, tablet sweets, chewable tablets, dust formulations, capsules, and granules. They can be produced by a method similar to that for the above described pharmaceutical compositions. Foods include beverages. Specific examples of beverages include, nutritional supplements (e.g., drinkable preparations), tea beverages (e.g., green tea, oolong tea and black tea), soft drinks, jelly drinks, sports drinks, milk drinks, carbonated drinks, fruit juice beverages, lactic acid bacteria beverages, fermented milk drinks, powder beverages, cocoa drinks, coffee beverages and purified water. Furthermore, foods may be prepared as spreads (e.g., butter, jam, furikake and margarine), mayonnaise, shortening, custard cream, dressings, breads, cooked rice, noodles, pasta, miso soup, tofu, milk, yoghurt, soup, sauce, sweets (e.g., biscuits, cookies, chocolate, candies, cake, ice cream, chewing gum and tablets) or the like.

In addition to quinones or culture products, other food materials to be used for production of foods or feedstuffs, various nutrients, various vitamins, minerals, amino acids, various fats and oils, dietary fibers, various additives (e.g., a taste component, a sweetener, an acidulant such as organic acid, a surfactant, a pH adjuster, a stabilizer, an antioxidant, a pigment and a flavor) or the like may also be compounded in foods. Also, quinones or culture products may also be compounded in generally eaten foods.

When quinones or culture products obtained according to the present invention are added to food compositions, the quinone or culture product content differs depending on the form of a food. In general, content ranges from 0.5% by mass to 80% by mass, preferably ranges from 1% by mass to 60% by mass and more preferably ranges from 3% by mass to 50% by mass, based on dry mass. The daily intake may be ingested all at once or in several separate doses. It is preferable to be able to control the daily intake, so that an adult can ingest the above described daily intake by eating or drinking.

Examples of foods include not only foods for humans, but also feedstuffs for domestic animals, race horses and the like, as well as pet foods. Feedstuffs are almost the same as foods, except that feedstuffs are intended for animals. Hence, descriptions concerning foods in this specification can be similarly applied to feedstuffs.

The present invention will be further described in detail by referring to the examples. However, the invention is not limited by these examples.

### EXAMPLES

Menaquinone in the culture products of the present invention was extracted and measured in the following Examples.

### Extraction of Menaquinone

A culture solution or a solution prepared by dissolving a biofilm in distilled water was centrifuged (12000 rpm, 4° C., 10 minutes), so as to obtain 2.5 mL of a supernatant. Isopropanol (2.5 mL) and 2.5 mL of hexane were added to the supernatant and then the mixture was stirred well. After stirring, the resultant was left to stand so that it separated into two layers. The upper layer (hexane layer) was recovered and then hexane was removed under reduced pressure conditions. These procedures were carried out under ice-cold and light-shielded conditions to the greatest extent possible.

### Measurement of Menaquinone

The culture product from which hexane had been removed as described above was dissolved in 200 μL of methanol, and a sample for measurement was thereby prepared. The amount of menaquinone was measured under the following conditions using a high performance liquid chromatography method. Measurement was carried out by reducing menaquinone so as to yield menaquinol and then using a fluorescence method.

Separation column: COSMOSIL 5C18-MS-II
Reduction column: Platinum-Alumina Column
Column temperature: 25° C.
Detector: Excitation wavelength 240 nm, detection wavelength 430 nm
Mobile phase: methanol:2-propanol=8:2
Flow rate: 1.0 mL/min

Menaquinone existing within cells that was not secreted extracellularly from microorganisms was also measured as follows.

Extraction and Measurement of Menaquinone within Cells

A culture solution or a solution prepared by dissolving a biofilm in distilled water was centrifuged (12000 rpm, 4° C., 10 minutes), followed by removal of a supernatant. One mL of 1% sodium chloride solution was added to the precipitate. The resultant was shaken, suspended, and then centrifuged (12000 rpm, 4° C., 5 minutes), followed by removal of the supernatant. One mL of sterile water, 2.5 mL of methanol and 1.25 mL of chloroform were added to the precipitate. The mixture was shaken and then left to stand. 1.25 mL of Chloroform was added, and then the resultant was shaken. 1.25 mL sterile water was added and then the resultant was shaken and then left to stand. The lower layer (chloroform layer) was recovered. One mL of chloroform was added to the upper layer (aqueous layer). The resultant was shaken and left to stand. The lower layer (chloroform layer) was recovered and then mixed with the previous chloroform layer. Chloroform was removed under reduced pressure conditions. Procedures were carried out under ice-cold and light-shielding conditions to the greatest extent possible. The amount of menaquinone was measured in a manner similar to that described above.

The amount of a biofilm formed on a porous carrier as a result of culture was measured as follows.

### Measurement of Biofilm Amounts

Culture was carried out in the presence of a porous carrier. After completion of culture, the porous carrier was removed. This was washed twice with distilled water and then immersed in 0.1% crystal violet solution for 25 minutes, so that the biofilm adhering to the porous carrier was stained. Excessive crystal violet solution was washed off. The thus stained porous carrier was immersed in 33% acetic acid and then a pigment was extracted. Absorbance of the extract was measured at 595 nm, and the amount of the biofilm was thereby found.

### Example 1

### Differences in Menaquinone Yield Resulting from the Presence or the Absence of Porous Carriers

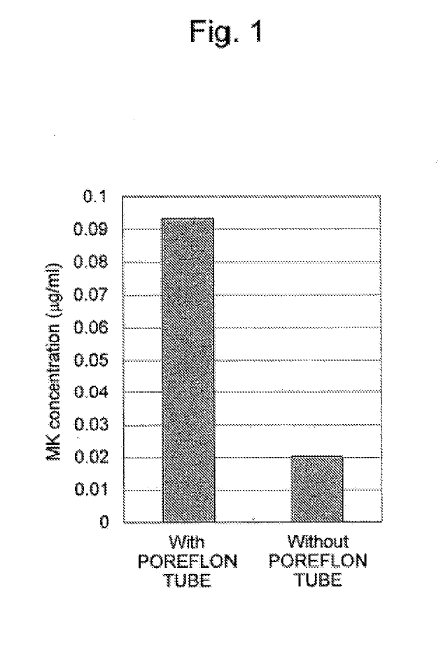

One polytetrafluoroethylene porous tube (Poreflon tube TB-0403; Sumitomo Electric Fine Polymer Inc.) cut to a length of 13 cm was placed in a 50-mL conical tube (Bioscience) as a porous carrier. Thirty mL of LB medium with the following composition was added, and then 300 μL of a solution (OD₆₀₀=0.3) prepared by diluting Bacillus subtilis with sterile water was introduced into the tube. After introduction, static culture was carried out for 24 hours at 37° C. As a control, culture was separately carried out using a culture solution alone into which no porous carrier had been added. After completion of culture, the amount of menaquinone (MK) in each culture solution was measured. FIG. 1 shows the results.

Medium composition (LB medium): Bacto Tryptone 10.0 g/L, yeast extract 5.0 g/L, sodium chloride 5.0 g/L, pH=7.3

As is understood from the results in FIG. 1, culture in the presence of a Poreflon tube which is the porous carrier resulted in a drastically increased amount of menaquinone produced.

### Example 2

### Differences in Yield Resulting from Biosurfactants

With the use of an experimental Bacillus subtilis strain 168 (ATCC 6051) (sfp− strain) producing no surfactin which is the biosurfactant and an sfp+strain prepared by introducing a surfactin synthesis gene (sfp) into the sfp− strain, the amounts of menaquinone produced were compared. The sfp+ strain was prepared introducing a plasmid vector in which the sfp gene had been incorporated into an sfp− strain. Culture was carried out in a manner similar to that in Example 1. The sfp+strain alone, the sfp− strain alone and a culture solution of the sfp− strain to which surfactin had been added were used.

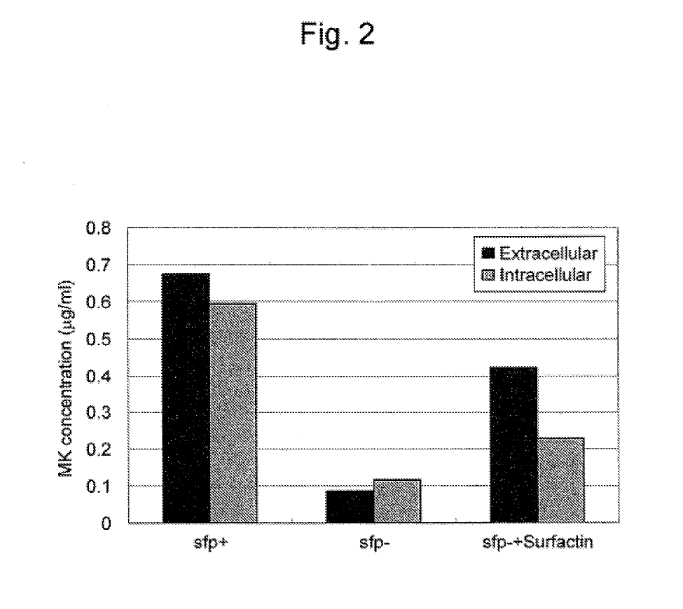

100 mL of LB medium prepared in a manner similar to that in Example 1 was added to a 500-mL conical tube (Bioscience) and then 1 mL of the sfp+strain and 1 mL of the sfp− strain were each introduced. Also, separately, 1 mL of the sfp− strain was introduced and then surfactin was dissolved to a final concentration of 116 μM (the same amount as the production amount of the sfp+strain). After introduction, shaking culture (120 rpm) was carried out for 16 hours at 37° C. After the completion of culture, intracellular and extracellular amounts of menaquinone in each conical tube (that is, the amount of menaquinone in each culture product) was measured. The results are shown in FIG. 2.

As is understood from the results in FIG. 2, addition of surfactin to a culture solution increased the amount of menaquinone (MK) produced to an extent greater than that of the sfp− strain producing no surfactin. In the case of the sfp+ strain producing surfactin, an even greater amount of menaquinone was produced and a large amount of menaquinone was produced and secreted not only within cells, but also in a culture solution, as shown in FIG. 2.

### Example 3

### Differences in Menaquinone Yield Resulting from Biofilms

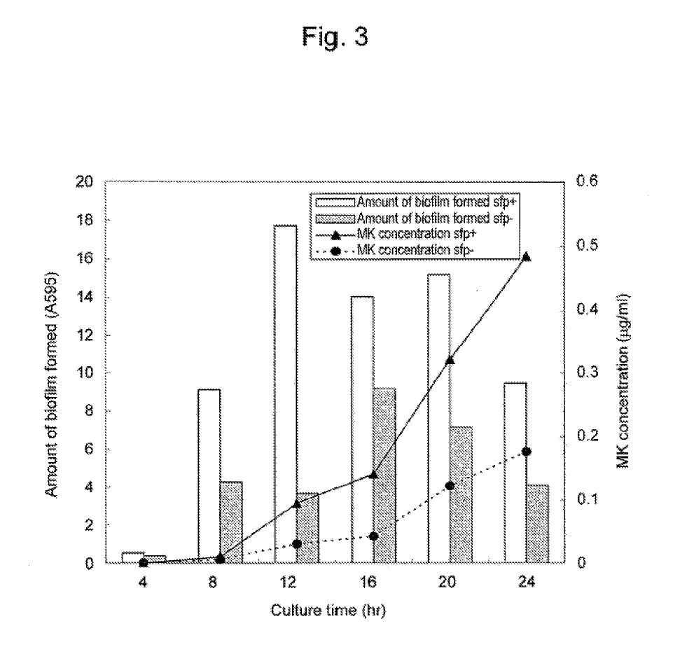

The sfp+ strain and the sfp− strain prepared in Example 2 were subjected to static culture under conditions similar to those in Example 1 in the presence of a porous carrier for 24 hours. The amounts of menaquinone produced were compared. At 4, 8, 12, 16, 20 and 24 hours after initiation of culture, the amount of menaquinone in each culture solution and the amount of biofilm formed were measured. The results are shown in FIG. 3.

As is understood from the results in FIG. 3, menaquinone yield was significantly higher in the sfp+ strain producing surfactin than in the sfp− strain producing no surfactin. Also, a biofilm was formed in a greater amount in the sfp+ strain producing surfactin than the sfp− strain producing no surfactin.

### Example 4

### Differences in Menaquinone Yield Resulting from Aeration Volumes

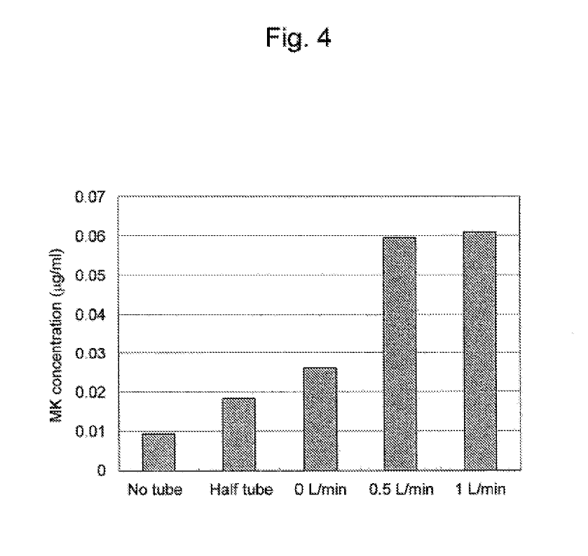

Effects resulting from aeration of a culture solution were examined. One polytetrafluoroethylene porous tube (Poreflon tube TB-0403; Sumitomo Electric Fine Polymer Inc.) cut to a length of 13 cm or 26 cm was placed in a 100-mL medium bottle as a porous carrier, 100 mL of LB medium was added to the bottle, and then 300 μL of a solution (OD₆₀₀=0.3) prepared by diluting Bacillus subtilis with sterile water was introduced into the bottle. A culture solution in a bottle containing a 26 cm-long Poreflon tube was aerated via a pump with air (45° C., humidity 90%) that had passed through an artificial climate chamber at flow rate of 0, 0.5 and 1 L/min. Static culture was carried out for 24 hours after introduction at 37° C. After completion of culture, the amount of menaquinone (MK) in each culture solution was measured. The results are shown in FIG. 4.

As shown in FIG. 4, the greater the extent of aeration, the higher the amount of menaquinone produced. Also, the use of the 26 cm porous tube resulted in higher production amounts of quinones than those in the case of the 13 cm porous tube.

### Example 5

### Differences in Menaquinone Yield Resulting from Culture Methods

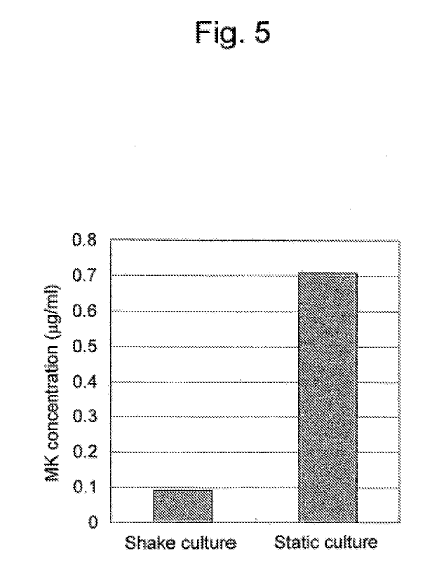

Effects due to the use of different culture methods (static culture and shaking culture) were examined. Two polytetrafluoroethylene porous tubes (Poreflon tube TB-0403; Sumitomo Electric Fine Polymer Inc.) each cut to a length of 13 cm were placed in separate 100 mL medium bottles as porous carriers. TBS medium (100 mL) with the following composition was added to the bottle and then 300 μL of a solution (OD₆₀₀=0.9) prepared by diluting Bacillus subtilis natto isolated from commercially available natto with sterile water was introduced. Each culture solution was aerated via a pump with air (45° C., humidity 90%) that had passed through an artificial climate chamber at a flow rate of 1 L/min. Static culture or shaking culture (at 120 rpm) was carried out for 24 hours after introduction at 37° C. The results are shown in FIG. 5.

Medium composition (TSB medium): TSB (Tryptic Soy Broth; Wako Pure Chemical Industries, Ltd.) 30.0 g/L, pH=7.4

As shown in FIG. 5, the production amount of menaquinone was higher in the case of static culture.

### Example 6

### Condensation of Culture Solutions

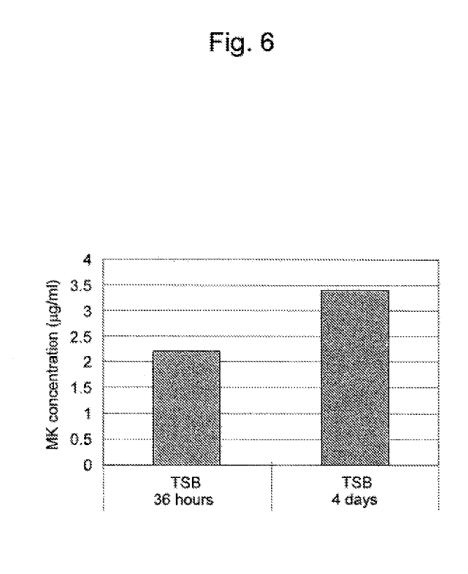

Upon aeration of a culture solution, the effects of aeration via a porous carrier were examined. Culture was carried out under conditions listed in Table 1. The results are shown in FIG. 6.

TABLE 1

| | Microorganism | |
|---|---|---|
| | Bacillus subtilis natto | Bacillus subtilis natto |
| | in Example 5 | in Example 5 |
| Volume of | 50 mL | 100 mL |
| culture solution | | |
| Culture solution | TSB | TSB |
| Culture temperature | 37° C. | 37° C. |
| Culture conditions | Static culture | Static culture |
| Porous carrier | POREFLON TUBE, | POREFLON TUBE, |
| | 13 cm | 13 cm × 2 |
| Aeration | 1 L/min | 1 L/min |
| Culture time | 36 hours | 4 days |

As is understood from FIG. 6, through aeration via the porous carrier, a culture solution could be condensed efficiently and a menaquinone-containing culture product with a high concentration could be obtained.

### Example 7

### Purification of Culture Solutions

1000 ml of water and 2000 ml of hexane were added to 500 g of the menaquinone-containing culture product prepared in Example 6, followed by 30 minutes of stirring. A hexane layer was recovered after stirring, so that a crudely purified menaquinone-containing culture product was obtained. The crudely purified culture product was then subjected to distillation for purification using a molecular distillation pilot apparatus. Distillation was carried out at 200° C. to 250° C., at supply flow rate kept between approximately 2 and 3 L/h, and with distillation pressure of 2×10⁻⁴ mbar. Thus, a residue and a distilled and purified product were obtained and the yields thereof were 72% and 18%, respectively (with menaquinone purity of 95% or higher). Therefore, it was understood that highly pure menaquinone can be conveniently obtained with the use of the culture product of the present invention.

What is claimed is:

1. A method for producing quinones, comprising culturing a microorganism producing quinones in the presence of a porous carrier.

2. The method according to claim 1, wherein the microorganism is a microorganism forming a biofilm.

3. The method according to claim 1, wherein the microorganism is a microorganism producing a biosurfactant.

4. The method according to claim 2, wherein the microorganism is a microorganism producing a biosurfactant.

5. The method according to claim 1, wherein the microorganism is a microorganism belonging to Bacillus.

6. The method according to claim 2, wherein the microorganism is a microorganism belonging to Bacillus.

7. The method according to claim 3, wherein the microorganism is a microorganism belonging to Bacillus.

8. The method according to claim 4, wherein the microorganism is a microorganism belonging to Bacillus.

9. The method according to claim 5, wherein the microorganism is Bacillus subtilis.

10. The method according to claim 6, wherein the microorganism is Bacillus subtilis.

11. The method according to claim 7, wherein the microorganism is Bacillus subtilis.

12. The method according to claim 8, wherein the microorganism is Bacillus subtilis.

13. The method according to claim 9, wherein the microorganism is Bacillus subtilis var natto.

14. The method according to claim 10, wherein the microorganism is Bacillus subtilis var natto.

15. The method according to claim 11, wherein the microorganism is Bacillus subtilis var natto.

16. The method according to claim 12, wherein the microorganism is Bacillus subtilis var natto.

17. The method according to claim 1, further comprising adding a biosurfactant to a culture solution.

18. The method according to claim 1, wherein a quinone is menaquinone.

19. The method according to claim 1, wherein culture is carried out with aeration.
